(19) United States
(12) Patent Application Publication
(10) Pub. No.: US 2013/0178931 A1
(43) Pub. Date: Jul. 11, 2013

(54) HEART VALVE PROSTHESIS

(71) Applicant: Biotronik AG, Buelach (CH)

(72) Inventor: Amir Fargahi, Buelach (CH)

(73) Assignee: Biotronik AG, Buelach (CH)

(21) Appl. No.: 13/710,719

(22) Filed: Dec. 11, 2012

Related U.S. Application Data
(60) Provisional application No. 61/585,273, filed on Jan. 11, 2012.

(51) Int. Cl. A61F 2/24

(52) U.S. Cl. CPC A61F 2/2442
USPC 623/2.36

(57) ABSTRACT

The invention relates to a heart valve prosthesis comprising biological material or plastic material, having a suture or knot which comprises an X-ray visible material and the position of which identifies the valve plane.

This application claims the benefit of U.S. Application Ser. No. 61/585,273, filed Jan. 11, 2012, entitled Heart Valve Prosthesis.

### TECHNICAL FIELD

The invention relates to a heart valve prosthesis comprising biological material or plastic material.

### BACKGROUND

Heart valve prostheses have been known and used in clinical applications for quite some time. For several years, versions have been available which are inserted in a vascular stent and can be introduced using minimally invasive surgical techniques, for example the products “Sapien” from Edwards Lifesciences or “Corevalve” from Medtronic Inc.

The open-heart insertion of a heart valve prosthesis is done under the direct visual monitoring of the surgeon, and the prosthesis can in particular be placed with a correct position of the heart valve plane, correct positioning during minimally invasive procedures is done solely based on X-ray monitoring. However, it has been found to be difficult to establish the position of the heart valve plane, whereby the reliable correct position of the prosthesis is also problematic.

### SUMMARY

It is therefore the object of the invention to provide an improved heart valve prosthesis, which can be easily and reliably positioned correctly, especially under X-ray monitoring.

One aspect of the invention encompasses providing an X-ray visible marking on the heart valve prosthesis. Another aspect encompasses avoiding the application of X-ray markers, notably known hard, planar markers, that complicate the design of the prosthesis and/or potentially impair the function. For this reason, a suture, or optionally a knot, comprising X-ray visible material (metal wire) is proposed.

### BRIEF DESCRIPTION OF THE DRAWINGS

Advantages and functional characteristics of the invention will additionally become apparent hereafter from the description of exemplary embodiments and aspects based on the figures. In the drawings:

FIG. 1 shows a schematic perspective illustration of an aortic heart valve prosthesis, for example, comprising essential sutures,

FIG. 2 is a perspective illustration of a heart valve prosthesis that can be implemented using a minimally invasive procedure, and

FIG. 3 is a detail of a connecting region between the actual heart valve and the vascular stent for a heart valve prosthesis that can be implemented using a minimally invasive procedure.

### DETAILED DESCRIPTION

When producing a suture, which is to be provided in the prosthesis anyway, from an X-ray visible material (metal wire), the function basically does not change and the manufacturing complexity does not increase considerably. It is thus possible to identify the heart valve plane with X-ray monitoring, without limiting the functional capability of the prosthesis, whereby the usability in clinical applications and the prospects in the market are improved. This can be also achieved in a design in which the suture comprising the metal wire doubles a section of a suture that is produced from a different suturing material.

In some embodiments, the metal wire comprises or is coated with at least one of the elements gold, platinum, tantalum and tungsten. Desirably, it comprises or is coated with at least one of gold, platinum or tungsten. In other embodiments, it is also possible to employ other metals as the suturing material, or at least as a component of a suturing material, such as stainless steel, or another white metal instead of platinum, or alloys comprising two or more of the aforementioned elements.

According to a further embodiment of the invention, the metal wire has a diameter ranging between about 20 and about 200 μm, and more particularly between about 50 and about 100 μm. When using an X-ray visible suturing material, which is not composed entirely of a metal wire, but in which a fine metal wire is twisted, for example, with conventional suture material, wire diameters close to the aforementioned lower limit, or even below that, are possible provided that sufficient visibility in the X-ray is preserved. In this context, according to further embodiments of the invention, the suture is implemented using the X-ray visible metal wire in the form of double, overlap, overlock or other visually noticeable suture.

In some embodiments, the invention can be implemented in a suture from X-ray visible material in which the course (longitudinal extension) corresponds in the known manner to the position of the valve plane and thereby identifies the same. In other embodiments, the invention can be implemented in designs in which the suture comprising the X-ray visible metal wire is placed such that a predefined end thereof identifies the position of the valve plane.

In a typical design of an aortic heart valve prosthesis, for example, comprising a base body, into which three valvular cusps are sewn, each with a suture, a suture section comprising an X-ray visible metal wire is provided on each valvular cusp such that the three suture sections together identify the valve plane. According to an alternative solution, in a heart valve prosthesis that is sewn into a vascular stent only the vascular stent contains a suture section comprising the X-ray visible metal wire, the position of which identifies the valve plane. Again, knots shall be understood to be included in the meaning of suture sections.

A further design of the present invention represents a mitral valve, in which two valvular cusps, as described above, have been sewn in using a respective suture comprising an X-ray visible metal wire.

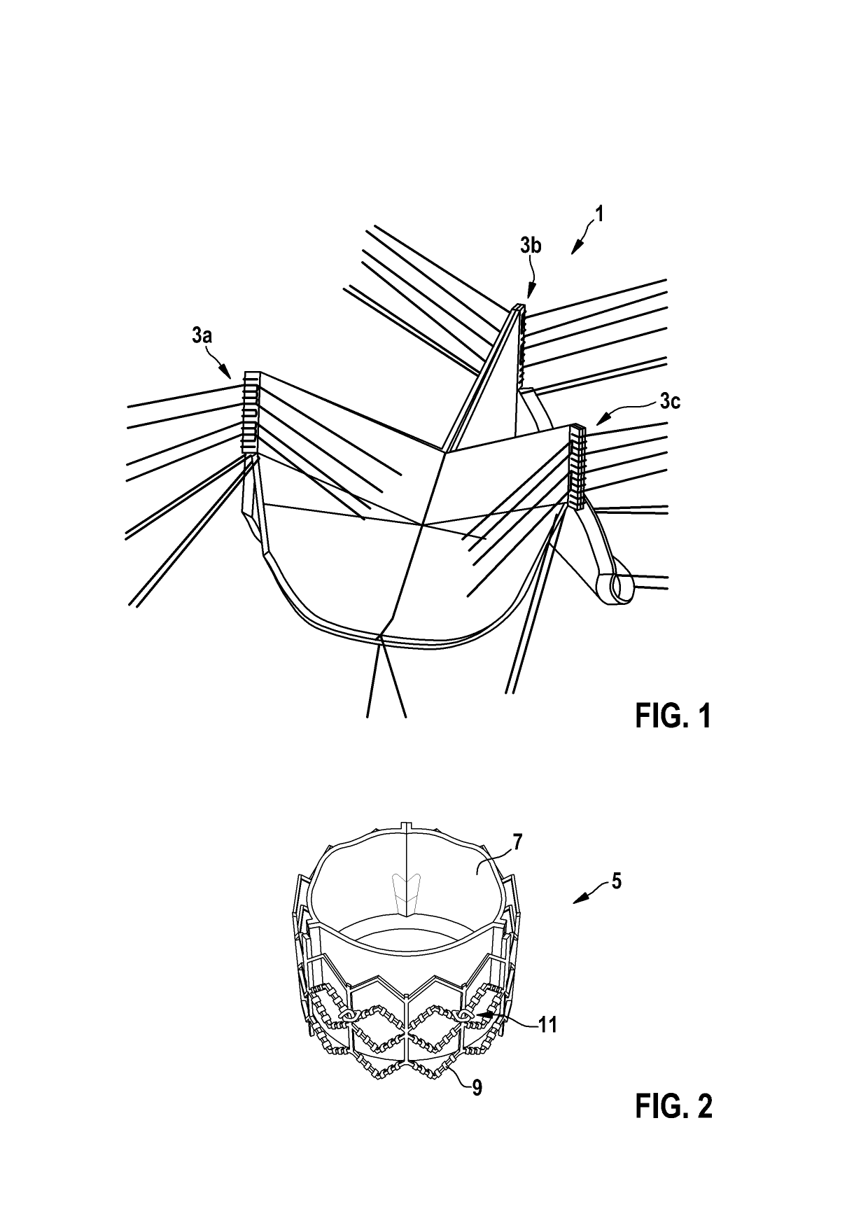

FIG. 1 shows an aortic valve prosthesis 1, which can be produced, for example, from pig, horse or bovine percardial tissue, comprising three suture sections 3a, 3b, 3c and several reference sutures or stitches (which are not denoted separately). Designing the suture regions 3a, 3b, 3c with an X-ray visible metal wire, for example made of platinum, renders the sutures visible under X-ray monitoring, giving the surgeon an indication of the position of the valve plane.

FIG. 2 shows a commercially available heart valve prosthesis 5 which can be implanted using a minimally invasive procedure and in which the actual heart valve 7 made of biological material is introduced in a stent 9. Knots 11 comprising X-ray visible material are shown by way of example, which when designed from sufficiently thick metal wire, and optionally with a suitable knot design, can be noticeably distinguished in the X-ray image from the lattice structure of the stent and thus allow an assessment of the position of the valve plane.

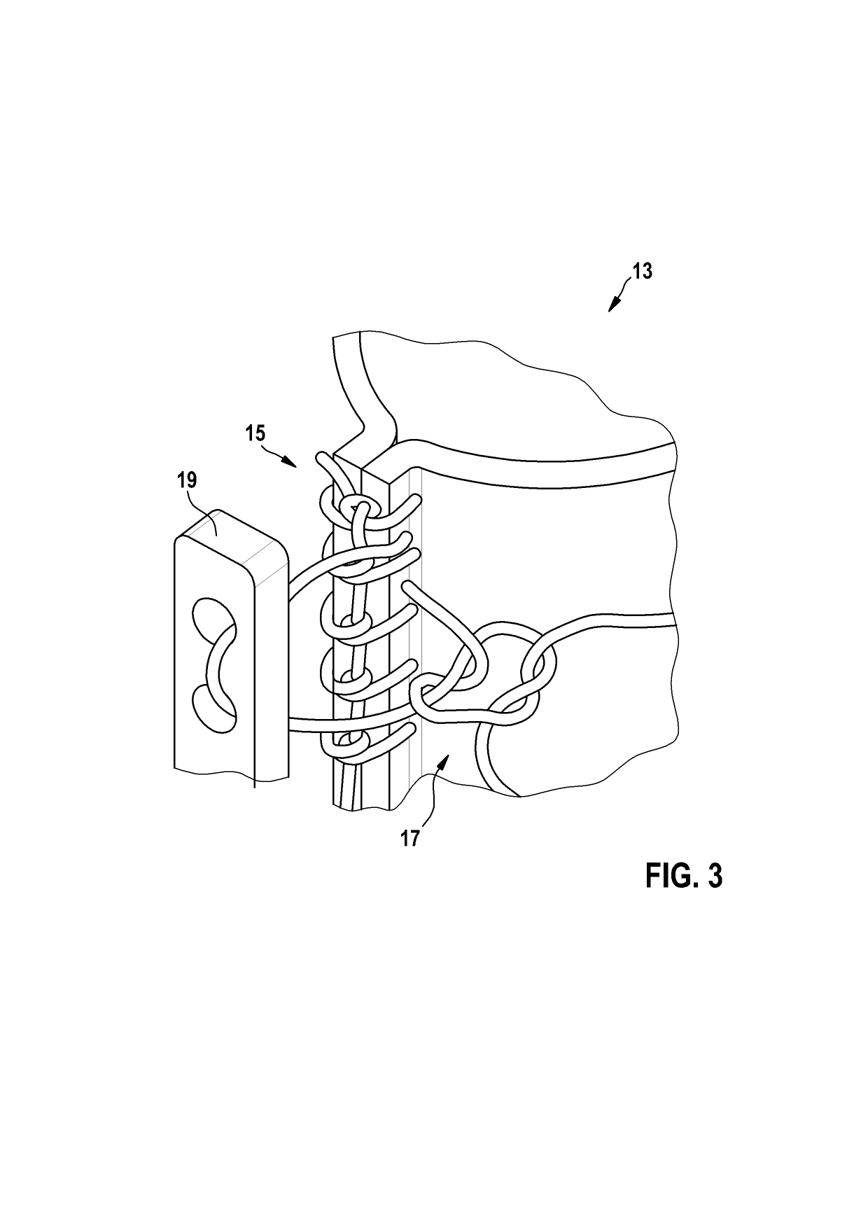

FIG. 3, which is a detail of a further heart valve prosthesis 13 that can be implanted using a minimally invasive procedure, shows a suture section 15, to which parts of the prosthesis are sewn, together with a further suture/knot section 17, to which the actual heart valve is attached on a fastening section 19 of an associated stent. When designing the suture section 17 from metal wire, it is again possible to notice the same on the X-ray image and thus draw a conclusion of the position of the valve plane.

The implementation of the invention is not limited to the examples described above and concepts emphasized, but is likewise possible in a plurality of modifications, which are within the scope of standard practice in the art.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A heart valve prosthesis made of biological material or plastic material, having a suture section including at suture or knot which comprises an X-ray visible metal wire wherein a position of the suture section identifies a plane of the heart valve prosthesis.

2. The heart valve prosthesis according to claim 1, wherein the metal wire comprises or is coated with at least one of the elements gold, platinum, tantalum and tungsten.

3. A heart valve prosthesis according to claim 1, wherein the metal wire has a diameter ranging between about 20 and about 200 μm.

4. A heart valve prosthesis according to claim 1, wherein the suture has a portion produced from a different suture material.

5. A heart valve prosthesis according to claim 1, wherein the suture comprises a double suture, an overlap suture, or an overlock suture.

6. A heart valve prosthesis according to claim 1, wherein the heart valve prosthesis comprises a base body, and three valvular cusps, each valvular cusp having the suture section.

7. A heart valve prosthesis according to claim 6, wherein the valvular cusps are sewn into the base body with the suture.

8. A heart valve prosthesis according to claim 1, wherein the suture is placed such that a predefined end thereof identifies a position of the valve plane.

9. A heart valve prosthesis according to claim 1, wherein the suture section includes the knot.

10. A heart valve prosthesis according to claim 1, which is sewn into a vascular stent, wherein a suture connecting the heart valve prosthesis to the vascular stent is arranged in a corresponding manner to a position of the valve plane and is produced from the X-ray visible metal wire.

11. A heart valve prosthesis according to claim 1, which is sewn into a vascular stent, wherein the vascular stent has a suture section which comprises the X-ray visible metal wire and a position of the suture section of the vascular stent identifies the valve plane.

12. A heart valve prosthesis according to claim 1, wherein the metal wire has a diameter ranging between about 50 and about 100 μm.

13. A heart valve prosthesis made of biological material or plastic material, the heart valve prosthesis comprising a base body, and three valvular cusps, each valvular cusp having a suture section including at suture or knot which comprises an X-ray visible metal wire, the valvular cusps being sewn into the base body with the suture, wherein a position of the suture sections identifies a plane of the heart valve prosthesis.

14. The heart valve prosthesis according to claim 13, wherein the metal wire comprises or is coated with at least one of the elements gold, platinum, tantalum and tungsten.

15. A heart valve prosthesis according to claim 13, wherein the metal wire has a diameter ranging between about 20 and about 200 μm.

16. A heart valve prosthesis according to claim 13, wherein the suture has a portion produced from a different suture material.

17. A heart valve prosthesis according to claim 13, wherein the suture comprises a double suture, an overlap suture, or an overlock suture.

18. A heart valve prosthesis according to claim 13, wherein the suture is placed such that a predefined end thereof identifies a position of the valve plane.

19. A heart valve prosthesis according to claim 13, which is sewn into a vascular stent, wherein a suture connecting the heart valve prosthesis to the vascular stent is arranged in a corresponding manner to a position of the valve plane and is produced from the X-ray visible metal wire.

20. A heart valve prosthesis according to claim 13, which is sewn into a vascular stent, wherein the vascular stent has a suture section which comprises the X-ray visible metal wire and a position of the suture section of the vascular stent identifies the valve plane.
